(19) United States
(12) Patent Application Publication
(10) Pub. No.: US 2025/0268702 A1
(43) Pub. Date: Aug. 28, 2025

(54) OXYGEN GENERATION SYSTEM AND APPLICATIONS THEREOF

(71) Applicants: NORTHWESTERN UNIVERSITY, Evanston, IL (US); CARNEGIE MELLON UNIVERSITY, Pittsburgh, PA (US)

(72) Inventors: Jonathan Rivnay, Chicago, IL (US); Itzhaq Cohen-Karni, Pittsburgh, PA (US); Inkyu Lee, Pittsburgh, PA (US); Abhijith Surendran, Evanston, IL (US); Seonghan Jo, Pittsburgh, PA (US); Xudong Ji, Evanston, IL (US)

(21) Appl. No.: 18/857,560

(22) PCT Filed: Apr. 20, 2023
(86) PCT No.: PCT/US2023/019215

§ 371 Date: Oct. 17, 2024

Related U.S. Application Data
(63) Continuation-in-part of application No. PCT/US2022/025686, filed on Apr. 21, 2022.
Application No. PCT/US2022/025686 is a continuation-in-part of application No. PCT/US2022/025706, filed on Apr. 21, 2022.
Application No. PCT/US2022/025706 is a continuation of application No. PCT/US2022/025724, filed on Apr. 21, 2022.
(60) Provisional application No. 63/332,876, filed on Apr. 20, 2022.
(60) Provisional application No. 63/419,461, filed on Oct. 26, 2022.
(60) Provisional application No. 63/177,806, filed on Apr. 21, 2021.

(51) Int. Cl. A61F 2/02; C12M 1/00; C12M 1/12; C25B 9/60; C25B 11/081

(52) U.S. Cl. CPC A61F 2/022; C12M 25/16; C12M 29/04; C25B 9/60; C25B 11/081

(57) ABSTRACT

An oxygenation system producing oxygen for therapeutic cells housed in an implanted or external device, the oxygenation system comprising a first electrode comprising a first material layer stack and a catalyst film of a high surface area or catalyst particles on which at least one catalyst for electrocatalytic oxygen evolution reaction is disposed; and a second electrode comprising a second material stack.

### CROSS-REFERENCE TO RELATED PATENT APPLICATION

This application claims priority to and the benefit of U.S. Provisional Patent Application Ser. No. 63/332,876, filed on Apr. 20, 2022, and U.S. Provisional Patent Application Ser. No. 63/419,461, filed on Oct. 26, 2022, which are incorporated herein by reference their entirety. This application is also a continuation-in-part application of and claims priority to the International Application No. PCT/US2022/025686, International Application No. PCT/US2022/025706, and International Application No. PCT/US2022/025724, which are concurrently filed on Apr. 21, 2022, and further claim priority to U.S. Provisional Patent Application Ser. No. 63/177,806, filed Apr. 21, 2021, which are also incorporated herein by reference their entirety.

### STATEMENT AS TO RIGHTS UNDER FEDERALLY-SPONSORED RESEARCH

This invention was made with government support under grant number FA8650-21-2-7119 awarded by the Air Force Research Laboratory. The government has certain rights in the invention.

### FIELD OF THE INVENTION

The present disclosure relates to the technical field of an oxygenation system producing oxygen for cell cultures via oxygen evolution reaction, and application of the system in combination with an implantable or wearable device.

### BACKGROUND OF THE INVENTION

The background description provided herein is for the purpose of generally presenting the context of the present invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

Oxygen evolution reaction (“OER”) is used in many applications such as electroextraction of metals and fuel generation using solar power. Controlled production of oxygen at point-of-use is of substantial importance in biotechnological and medical applications. An emerging application is to supply oxygen to biological cell cultures or tissues in order to sustain cell health/metabolic function, and/or to enable cell densities which is too dense to be maintained without vascularization. Maintenance of cell viability and function is essential and fundamentally limited by nutrients and oxygen supplies. Bochenek et al (Nature Biomedical Engineering 2018) demonstrated that oxygen levels in vivo vary between 20 torr to 60 torr, depending on tissue type, location and level of vascularization.

Thus, local and integrated O₂ production is important for a range of applications from in vitro bioreactors to implantable in vivo device. This includes applications where cell homo cultures, multi-cell assemblies, organoids or engineered tissues need to maintain high viability and productivity at high cell densities, or in vivo, where native vascularization is impossible due to suppression of immune responses. Possible embodiments include, but are not limited to, islet encapsulation, artificial pancreas, and oxygenation device incorporated in engineered tissues for local delivery of oxygen.

Oxygen diffusion limitations may be overcome by engineering immunobarrier (membrane or hydrogel encapsulation) layer diffusion coefficients and/or modifying the diffusion distances between the device and a host tissue. However, this approach significantly limits the minimum device footprint and/or cell density. On the other hand, providing exogenous oxygen, which leads to a direct pO₂ increase adjacent to the cell culture, may circumvent the described limitations. However, some of these approaches rely on external tubing to relay external O₂ gas through a bioreactor/tissue, complicating the implementation.

Therefore, there remains an imperative need for an oxygenation system which could provide long-term localized in vitro and/or in-vivo oxygen production, so as to generate oxygen for cell cultures and other oxygen-based bioactivities at the point-of-use.

### SUMMARY OF THE INVENTION

This work establishes a generalizable engineering framework of an oxygenation system for providing oxygen to cell cultures or other oxygen dependent environments/bioactivities in both in vivo and in vitro embodiments.

In light of the foregoing, this invention discloses an oxygenation system producing oxygen via electrocatalytic OER, and the oxygen delivery can be highly localized, multisite, and readily and precisely controlled.

This disclosure also discloses an oxygenation system which is integrated with a fully implanted or external device having a cell containment system housing living cells that are engineered to produce biomolecules constitutively, on external biophysical or biochemical trigger, or via genetically engineered sensing circuits (broadly considered as “cell therapies”).

This disclosure also discloses an oxygenation system which provides a uniform oxygen diffusion profile to the cell containment system.

In one embodiment of the invention, the oxygenation system may operate at a low overpotential which limits electrocatalytic production of unintentional side products. When high overpotentials are required for higher O₂ production, in order to protect the cell cultures or other oxygen dependent environments/bioactivities from side products produced by the oxygenation system, a membrane is incorporated surround or on top of the electrocatalyst and/or electrodes.

In one embodiment of the invention, the oxygenation system of the present invention exhibits excellent long-term stability, and may provide oxygen to cell cultures or other oxygen dependent environments/bioactivities for months or years without regular maintenance.

In one aspect of the invention, an oxygenation system for producing oxygen for therapeutic cells housed in a cell containment system, the cell containment system having a house containing the therapeutic cells and a nutrition solution comprising water, the oxygenation system comprising a first electrode comprising a first material layer stack and a catalyst film or catalyst particles of a high surface area on which at least one catalyst for electrocatalytic oxygen evolution reaction (OER) is disposed, wherein the first material layer stack has a series of transition metal layers stacked on one another, or a nanocarbon layer disposed on the stacked transition metal layers, and the catalyst film or catalyst particles are disposed on a top of the first material layer stack and vertically overlap with the first material layer stack; and a second electrode comprising a second material layer stack, wherein the second material layer stack has a series of transition metal layers stacked on one another, wherein the first electrode is coupled with the house and the second electrode is disposed in adjacent to the first electrode; wherein the catalyst for the electrocatalytic OER comprises at least one of ruthenium, iridium oxides, cobalt-based materials, nickel-based materials, organic-inorganic hybrids, and metalloprophyrin; and wherein the catalyst is at least partially in contact with the water of the nutrition solution.

In one embodiment, the oxygenation system further comprises a semi-permeable selective membrane disposed at an interaction surface between the first electrode and the nutrition solution.

In one embodiment, the semi-permeable selective membrane selectively permits the passage of water and oxygen and prevents the evolution of chloride.

In one embodiment, the first electrode and the second electrode have a design of two-point source grid topology.

In one embodiment, the first electrode and the second electrode have a design of two concentric ring topology.

In one aspect of the invention, an oxygenation system for producing oxygen for therapeutic cells housed in a house of a cell containment system, the oxygenation system comprising a first electrode comprising a first material layer stack, and a catalyst film or catalyst particles of a high surface area on which at least one catalyst for electrocatalytic oxygen evolution reaction (OER) is disposed; and a second electrode comprising a second material layer stack, wherein the first electrode is coupled with the house and the second electrode is disposed in adjacent to the first electrode.

In one embodiment, the first material layer stack comprises a series of transition metal layers stacked on one another, or a nanocarbon layer disposed on the stacked transition metal layers.

In one embodiment, the second material stack comprises a series of transition metal layers stacked on one another.

In one embodiment, the catalyst film or catalyst particles are disposed on a top of the first material layer stack and vertically overlaps with the first material layer stack partially or fully.

In one embodiment, the catalyst for the electrocatalytic OER comprises at least one of ruthenium, iridium oxides, cobalt-based materials, nickel-based materials, organic-inorganic hybrids, and metalloprophyrin.

In one embodiment, the catalyst is in contact with water of nutrition solution.

In one embodiment, the oxygenation system further comprises a semi-permeable selective membrane disposed at an interaction surface between the first electrode and the nutrition solution.

In one embodiment, the semi-permeable selective membrane selectively permits the passage of water and oxygen and prevents the evolution of chloride.

In one embodiment, a duty cycle of at least one of the first and second electrodes is adjustable.

In one embodiment, the first electrode and the second electrode have a design of two-point source grid topology.

In one embodiment, the first electrode and the second electrode have a design of two concentric ring topology.

In one aspect of the invention, an implanted or external device for housing therapeutic cells and producing oxygen for the therapeutic cells, the implanted or external devices comprising a cell containment system having a house containing the therapeutic cells and a nutrition solution comprising water; and an oxygenation system, wherein the oxygenation system comprises at least one electrode coupled with the house.

In one embodiment, at least one electrode comprises a first electrode and a second electrode.

In one embodiment, the first electrode comprises a catalyst film or catalyst particles of a high surface area on which at least one catalyst for electrocatalytic oxygen evolution reaction (OER) is disposed.

In one embodiment, the first electrode comprises a first material layer stack.

In one embodiment, the first material layer stack having a series of transition metal layers stacked on one another, or a nanocarbon layer on of the stacked transition metal layers stacked.

In one embodiment, the second electrode comprises a second material layer stack having a series of transition metal layers stacked on one another.

In one embodiment, the catalyst film is disposed on a top of the first material layer stack and vertically overlaps with the first material layer stack partially or fully.

In one embodiment, the catalyst for the electrocatalytic OER comprises at least one of ruthenium, iridium oxides, cobalt-based materials, nickel-based materials, organic-inorganic hybrids, and metalloprophyrin.

In one embodiment, the catalyst is in contact with water of the nutrition solution.

In one embodiment, the oxygenation system further comprises a first semi-permeable selective membrane disposed at an interaction surface between the first electrode and the nutrition solution.

In one embodiment, the first semi-permeable selective membrane selectively permits the passage of water and oxygen, and prevents the passage of cells, macromolecules, and salts, wherein the salts contain chloride ions.

In one embodiment, the implanted or external device further comprises a second semi-permeable selective membrane disposed at an interface between the implanted or external device and body environment of a user.

In one embodiment, the second semi-permeable selective membrane prevents passage of the immune cells of the user into the house.

In one embodiment, the oxygenation system comprises an insulating layer protecting the at least one electrode.

In one embodiment, the oxygenation system comprises a flexible substrate on which the at least one electrode is disposed.

In one embodiment, the therapeutic cells are encapsulated in a 3D morphology or hydrogel.

In one embodiment, a density of the therapeutic cells exceeds 50 k cells/mm³.

In one embodiment, a concentration of the produced oxygen in the nutrition solution is adjustable by controlling a duty cycle of the at least one electrode.

In one embodiment, the cell containment system comprises a light source configured to produce light for the therapeutic cells; wherein the light source is configured to produce a first light and a second light, and the second light having a wavelength different from the first light.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

Illustrative embodiments of the invention will hereafter be described with reference to the accompanying drawings, wherein like numerals denote like elements.

FIGS. 1A-B depict sectional views of oxygen generation systems in different embodiments. FIG. 1A depicts an embodiment of the invention showing in vitro oxygenation device for high-density therapeutic cell culture. FIG. 1B depicts another embodiment of the invention targeting the application for implantable therapeutic cell factories.

FIGS. 2A-B illustrate linear sweep voltammetry (LSV) showing examples of different electrodes and decorating catalysts materials for the electrocatalytic OER with various electrode dimensions; FIG. 2A depicts an embodiment using microelectrodes, and FIG. 2B depicts an embodiment using macroelectrodes.

FIG. 3 depicts topologies of simulation-guided electrode designs in row A, B, C, and D respectively; Column (I) shows estimated oxygen profiles of each design; Column (II)-(IV) reflect electrode geometries and magnified features with dimensions.

FIGS. 4A-B illustrate oxygen concentration profiles of the design electrodes, showing the feasibility of electrochemical oxygenation; FIG. 4A shows the chronopotentiometry of the design D as illustrated in FIG. 3; FIG. 4B shows chronoamperometry with 2-electrode set up of the design B as illustrated in FIG. 3.

FIGS. 5A-E provide an example of ion-selective polymeric membrane applied electrode; FIG. 5A-C show the long-term chronoamperometry measurement and generated oxygen profiles; FIG. 5D shows the chlorine detection test results at various potential; and FIG. 5E shows the LSV curves of the ion-selective polymeric membrane applied electrode.

FIGS. 6A-B provide evidence of in vitro cell maintenance under hypoxia (1% O2, 7.6 Torr) using the electrochemical oxygenation system; FIG. 6A shows 3D reconstruction of z-stacked images in the upper panel, and their maximum intensity projection images in the lower panel. Col. I shows positive control (normoxia), Col. II shows negative control (hypoxia), Col. III shows hypoxia+electrocatalytically oxygenated samples. FIG. 6B shows live/dead cell assay results of positive control, negative control and oxygenated condition.

FIG. 7 illustrates the generated oxygen partial pressure by applying different duty cycles with 1.7V potential.

FIGS. 8A-D illustrate oxygen concentration vs time at the various applied potentials/currents. FIG. 8A shows measured voltage profiles for various pulsed chronopotentiometry currents in one embodiment over the time; FIG. 8B shows the oxygen concentration of the electrode in FIG. 8A; FIG. 8C shows measured voltage profiles for various pulsed chronopotentiometry currents in another embodiment over the time; FIG. 8D shows the oxygen concentration of the electrode in FIG. 8C.

FIGS. 9A-C illustrate in vitro cell viability under different oxygen concentration level. FIG. 9A shows immunostaining of the cells under different oxygen concentration level; FIG. 9B shows the cell viability under different oxygen concentration level; FIG. 9C shows the leptin concentration over the time under different oxygen concentration level.

FIGS. 10A-B illustrate in vivo cell viability of a 10-day in vivo cell implantation with the oxygenation system and its control. In particular, FIG. 10A illustrates immunofluorescence images of a live/dead assay of the 10-day in vivo cell implantation; FIG. 10B illustrates statistics of the live/dead assay of 10-day in vivo cell implantation and its control.

### DETAILED DESCRIPTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

As used in the disclosure, the term “oxygenation” refers to an oxidation reaction wherein at least one oxygen atom is inserted into the molecule to be oxidized during the reaction.

As used in the disclosure, the term “oxygen evolution reaction” refers to a limiting reaction in the process of generating molecular oxygen through chemical reaction, such as the oxidation of water during oxygenic photosynthesis, electrolysis of water into oxygen and hydrogen, and electrocatalytic oxygen evolution from oxides and oxoacids.

As used in the disclosure, the term “high surface” refers a surface having a surface to volume ratio higher than the surface to volume ratio of a flat surface.

As used in the disclosure, the “cell culture(s)” refers to the maintenance or growth of natural or engineered cells under a set of controlled physical conditions.

As used in the disclosure, the term “implantable” refers to an ability of a device to be positioned at a location within a body, such as subcutaneously, within a body cavity, or etc. Furthermore, the terms “implantation” and “implanted” refer to the positioning of a device at a location within a body, such as subcutaneously, within a body cavity, or etc.

As used in the disclosure, “biocompatible” material is a material that is compatible with living tissue or a living system by not being toxic or injurious and not causing immunological rejection.

As used in the disclosure, “therapeutic agent” refers to any substance that provides therapeutic effects to a disease or symptom related thereto. In certain embodiments, a therapeutic agent refers to a substance that provides therapeutic effects to any diseases or biological or physiological responses to the diseases.

As used in the disclosure, the term “therapy” refers to any protocol, method, and/or agent that can be used in the management, treatment, and/or amelioration of a given disease, or a symptom related thereto. In certain embodiments, the terms “therapies” and “therapy” refer to a biological therapy, supportive therapy, and/or other therapies known to one of skill in the art, such as medical personnel, useful in the management or treatment of a given disease, or symptom related thereto.

As used in the disclosure, “treat,” “treatment,” and “treating” refer to the reduction or amelioration of the progression, severity, and/or duration of a given disease resulting from the administration of one or more therapies (including, but not limited to, the administration of microspheres disclosed herein). In certain embodiments, the terms refer to the reduction of pain associated with one or more diseases or conditions.

As used in the disclosure, “engineered cell(s)” refers herein to cells having been engineered, e.g. by the introduction of an exogenous nucleic acid sequence or specific alteration of an endogenous gene sequence. An exogenous nucleic acid sequence that is introduced may comprise a wild type sequence of any species that may be modified. An engineered cell may comprise genetic modifications such as one or more mutations, insertions and/or deletions in an endogenous gene and/or insertion of an exogenous nucleic acid (e.g. a genetic construct) in the genome. An engineered cell may refer to a cell in isolation or in culture. Engineered cells may be “transduced cells” wherein the cells have been infected with e.g. an engineered virus. For example, a retroviral vector may be used, such as described in the examples, but other suitable viral vectors may also be contemplated such as lentiviruses. Non-viral methods may also be used, such as transfections or electroporation of DNA vectors. DNA vectors that may be used are transposon vectors. Engineered cells may thus also be “stably transfected cells” or “transiently transfected cells”. Transfection refers to non-viral methods to transfer DNA (or RNA) to cells such that a gene is expressed. Transfection methods are widely known in the art, such as calcium phosphate transfection, PEG transfection, and liposomal or lipoplex transfection of nucleic acids. Such a transfection may be transient, but may also be a stable transfection wherein cells can be selected that have the gene construct integrated in their genome.

As used in the disclosure, “high surface area” refers to a structure and/or material having a high surface area to volume ratio. The surface-area-to-volume ratio is the amount of surface area per unit volume of an object or collection of objects. Structures and/or materials with high surface area may comprise features such as very small diameter, very porous, or otherwise not compact, and thus react at much faster rates than monolithic materials, because more surface is available to react.

An implanted or external device having a cell containment system housing encapsulated cell factories/therapy cells which produce therapeutic agents to a patient typically has a high-density cell loading. The implantable or external device is disclosed in International Application No. PCT/US22/25686, International Application No. PCT/US22/25706, and International Application No. PCT/US22/25724, to which the present application claims priority, are incorporated herein by reference in their entireties.

To enable the proposed high-density cell loading for the implanted or external device, oxygen can be supplied to the encapsulated cell factories or therapy cells. This can be accomplished via electrolysis of water at adjacent microelectrodes in one embodiment. In another embodiment, O₂ gradients are tailored and maintained to optimize the performance of the encapsulated cell factories/therapy cells by precisely tuning the platform's electrode size, spacing, and power supply. The use of selective polymer membranes can be used to minimize reactive side products and protect the device from bio-fouling. A primary goal of the present invention is to generate enhanced oxygen concentration on a small device footprint and with a low overall power budget. In alternative embodiments, depending on the type of cell(s) being used or the cell density, O₂ may be supplied with oxygen producing CaO nanoparticles.

In one embodiment, the present invention is directed, but not limited, to an oxygenation system integrated or used in combination with an implantable or wearable device. The implantable or wearable device comprises a cell-containment system housing encapsulated cells, and the oxygenation system having a microelectrode/macroelectrode structure which produces oxygen for encapsulated cells. In another embodiment, the oxygenation system is integrated with an engineered tissue, providing oxygen to the engineered tissue during its operation.

In one embodiment of the invention, the oxygenation system comprises (i) an electrode pair forming an electrochemical device for oxygen production, (ii) passivation layer to protect the electrodes, (iii) a semipermeable membrane to prevent Cl₂ or other side products evolution. The implantable or wearable device, in addition to the oxygenation system, may also comprise (iv) a cell containment system which houses the encapsulated cells.

In one embodiment, the oxygenation system produces oxygen via OER.

Production of oxygen via water splitting requires transfer of 4 electrons and 4 protons through a multistep reaction mechanism. Thus, significant overpotential is required to achieve necessary oxygen production. This sluggish OER kinetics is due to the energy losses involved in the formation of intermediary adsorbates on the anode surface. Therefore, the slow reaction kinetics calls for highly efficient electrocatalyst for the water splitting processes.

In one embodiment, ruthenium and iridium oxides are used as electrocatalysts for their excellent low overpotentials for the OER. In another embodiment, various transition metal compounds such as cobalt-based and/or nickel-based materials as well as organic-inorganic hybrids, for instance, metalloprophyrin and metal-organic frameworks (MOFs) are used as efficient catalysts for the OER. In one embodiment, these catalysts are used to decorate conductive materials and form electrocatalytic arrays. In one embodiment, the electrocatalysts for the OER are disposed on a film having a high surface area.

In one embodiment, the oxygen delivery by the oxygenation system of the present invention can be highly localized, multisite-based and readily controlled. Thus, the oxygenation system exhibits excellent long-term stability.

In one embodiment, the oxygenation system can operate at a low overpotential which limits electrocatalytic production of unintentional side products, e.g. Cl₂ gas.

Electrocatalysts can change the OER behaviors of pristine electrodes with varied geometries. Since catalytic materials can be deposited on conducting substrates through diverse techniques such as electrodeposition, sputtering, hydrothermal synthesis and chemical (either primary or secondary) interaction, the morphology of decorating materials can be tuned, allowing the electrocatalysts to interface reactants properly. In one embodiment, iridium oxide, such as EIROF, can be used as the electrocatalyst for OER and is electrodeposited or sputtered. In other embodiments, similar electrocatalysts are used on high surface area templates based on 3DFG, NT-3DFG, platinum or other suitable materials for electrode.

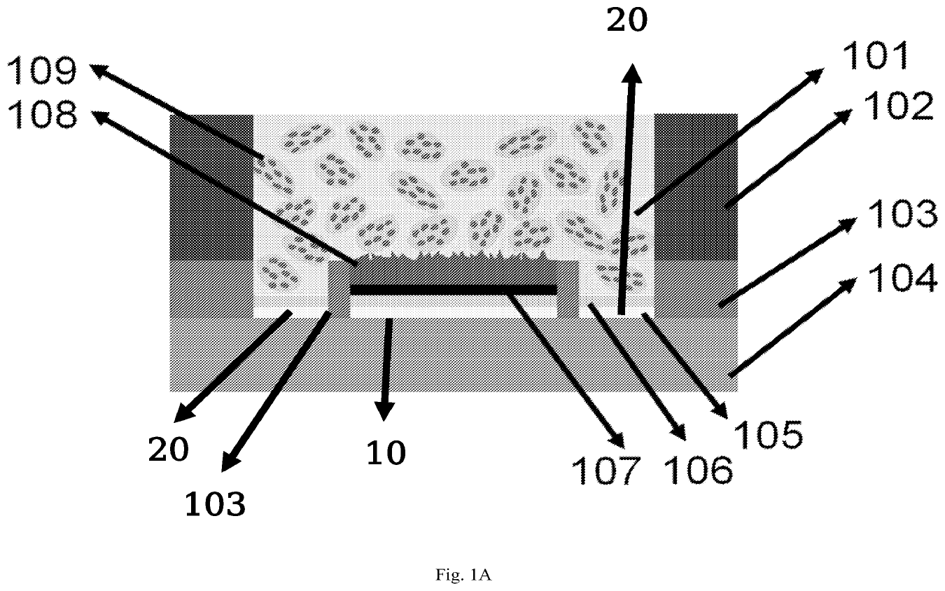

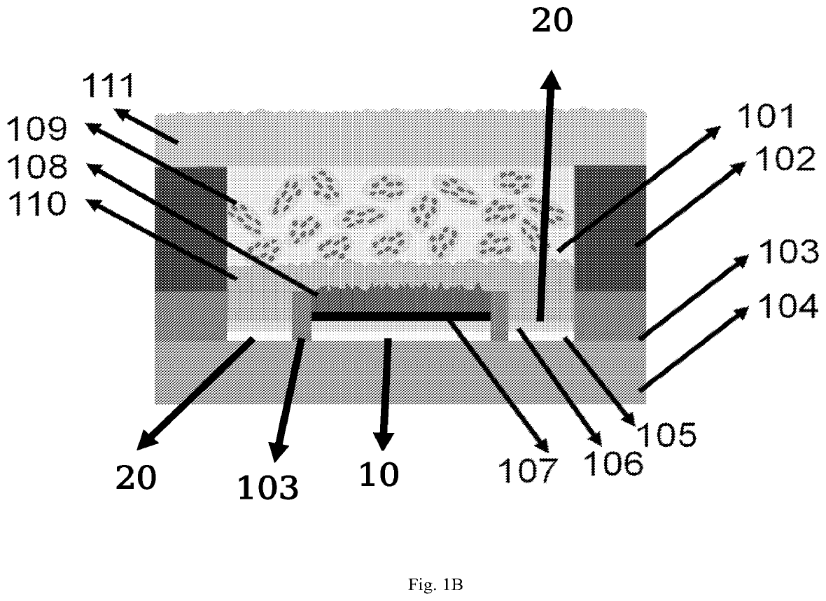

FIGS. 1A-B show a number of embodiments of the oxygenation system. Cross-sectional view of an embodiment of the invention is shown in FIG. 1A. The device has a 3D printed, machined or molded chamber 102 to contain nutrient solution 101 for natural or engineered cells which may produce therapeutic agents (“therapeutic cells”). In one embodiment, the therapeutic cells are encapsulated in a 3D morphology or hydrogel (e.g. alginate capsules) 109. The chamber 102 is designed to support high density of cell culture exceeding 50 k cells/mm³.

In one embodiment, the density of the cell culture supported by the chamber 102 and the oxygenation device exceeds 30K cells/mm³. In one embodiment, the density of the cell culture supported by the chamber 102 and the oxygenation device exceeds 40K cells/mm³. In one embodiment, the density of the cell culture supported by the chamber 102 and the oxygenation device exceeds 60K cells/mm³. In one embodiment, the density of the cell culture supported by the chamber 102 and the oxygenation device exceeds 70K cells/mm³. In one embodiment, the density of the cell culture supported by the chamber 102 and the oxygenation device exceeds 80K cells/mm³. In one embodiment, the density of the cell culture supported by the chamber 102 and the oxygenation device exceeds 90K cells/mm³.

Oxygen for the cells is provided through oxidation of water using a catalyst film having high surface area 108 at a working electrode 10.

In one embodiment, the device and the chamber may be supported using glass, silicon dioxide, or other flexible substrates 104. The working electrode 10 is more stable for long term use by using a metal stack. In one embodiment, a Ti(105)/Pt(106)/Ir(107) metal stack is used as the working electrode 10, whereas a counter electrode 20 has Ti(105)/Pt(106) stack. The electrical contacts are passivated using an insulating layer 103. In one embodiment, the insulating layer 103 is a hard-baked SU8 layer. In one embodiment, the working electrode 10 and the counter electrode 20 are separated by the insulating layer 103.

In one embodiment, the working electrode 10, as a first electrode, includes a material layer stack having a series of transition metal layers stacked on one another, or a nanocarbon layer on the stacked transition metal layers. In one embodiment, the counter electrode 20, as a second electrode, includes a material stack having a series of transition metal layers stacked on one another.

In one embodiment, the material layer stack of the working electrode 10 contains a series of transition metal layers or a nanocarbon layer on a series of transition metal layers. In one embodiment, the metal stack of the counter electrode 20 contains a series of transition metal layers.

In one embodiment, the working electrode 10 has a catalyst film of high surface area or catalyst particles of high surface area 108, on which one or more of the electrocatalysts for the OER are disposed. In one embodiment, the catalyst film 108 is disposed on a top of the material layer stack 105/106/107, and overlaps with the material layer stack 105/106/107 of the working electrode 10, partially for fully, as shown in FIG. 1A.

In one embodiment, as shown in FIG. 1A, the working electrode 10 and the counter electrode 20 are horizontally arranged on the substrates 104.

FIG. 1B discloses another embodiment of the present invention, which shows a cross-sectional view. In this embodiment, in addition to the elements disclosed in FIG. 1A, a semipermeable, selective membrane 110 is used to prevent the formation of side products such as Cl₂ gas at the working electrode 10 when the working electrode is at high overpotentials. In one embodiment, the semipermeable, selective membrane 110 comprises nafion. The membrane prevents Cl-ions being transported to the working electrode 10, while allowing the transportation of water molecules to the working electrode 10 for oxidation. In one embodiment, this embodiment of FIG. 1B is integrated with the implantable or wearable device housing encapsulated cells producing therapeutic agents, such that the implantable or wearable device is adapted for long-term use. Significant reduction of Cl₂ gas and other side products improves the safety of the oxygenation system and the cell containment system, and makes the implantable system suitable for being implanted into the body of a patient for long term use without side products related issues.

In one embodiment, prior to the implantation of the implantable or wearable device into a patient, the cell containment system is loaded with the therapeutic cells and may be covered using semipermeable membrane 111 to isolate the cells from the body's immune response. Thus, the semipermeable membrane 111 enhances the effectiveness and longevity of the therapeutic cells by shielding the cells from the immune response of the patient.

In another embodiment, either the oxygenation system in FIG. 1A or the oxygenation system in FIG. 1B is embedded within tissues to supply oxygen to implanted endogenous or exogenous cells.

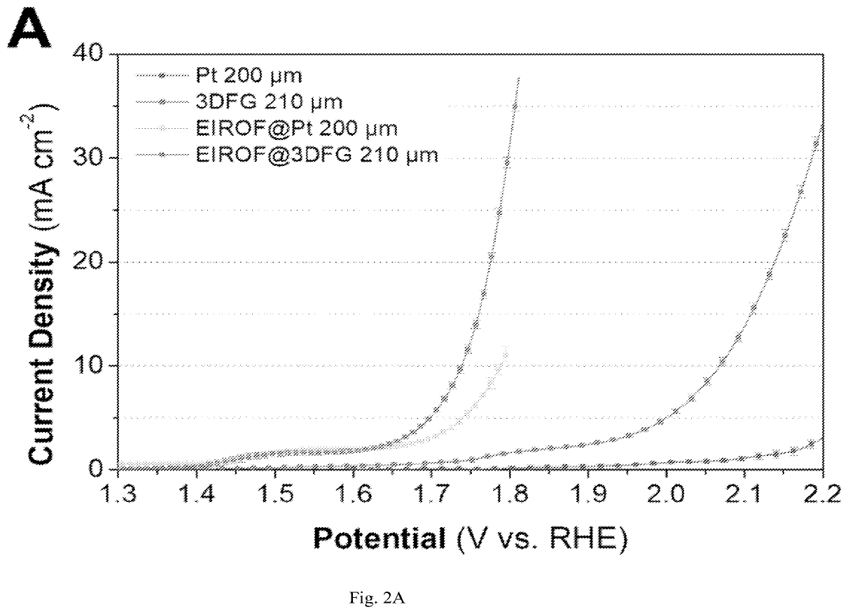

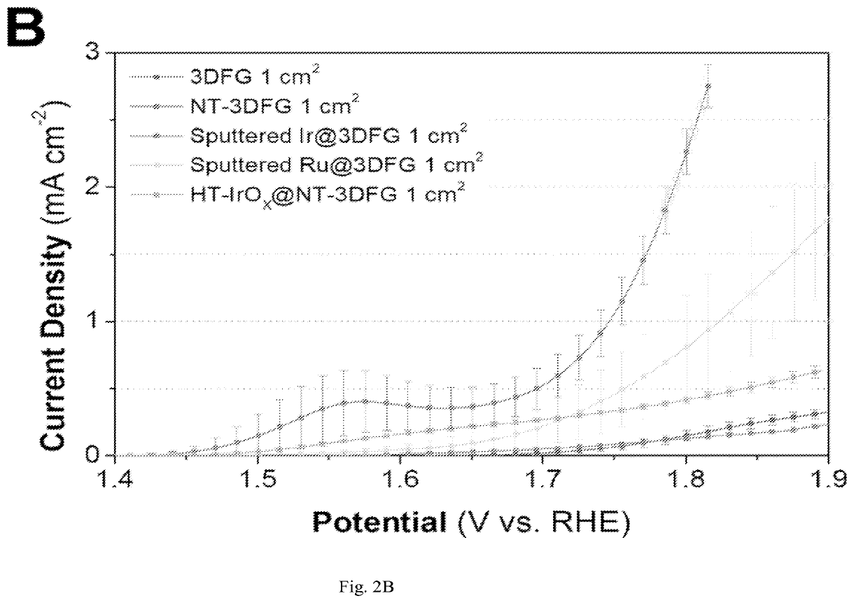

FIG. 2 depicts linear sweep voltammetry (LSV) showing a few examples of materials for electrodes and decorating catalysts used in the electrocatalytic OER with various dimensions. FIG. 2A shows microelectrodes having a size ranges approximately 200-210 μm and FIG. 2B shows macroelectrodes having a size ranges approximately 1 cm². Materials for the electrodes includes Pt, 3DFG, and NT-3DFG, and the decorating catalysts include EIROF, HT-IrOX, and etc. 3DFG represents 3-dimensional fuzzy graphene, NT-3DFG represents nanowire-template 3DFG, EIROF represents electrodeposited iridium oxide film, and HT-IrOX represents hydrothermally synthesized iridium oxide.

In one embodiment of the oxygenator system, the electrodes have designed geometries which are chosen such that the oxygen diffusion profile is uniform in the present invention. Simulation of the oxygen diffusion shows uniform oxygen concentration at different distances from the electrodes. In addition, in order to prevent massive hydrogen bubble formation due to high current density, the area ratio between the working and counter electrodes was tuned to enlarge the fraction of the counter electrode as much as possible, minimizing compromise on the working electrode side. When it comes to the working electrodes, each feature was optimized in terms of the number of windows on the working electrode and dimension.

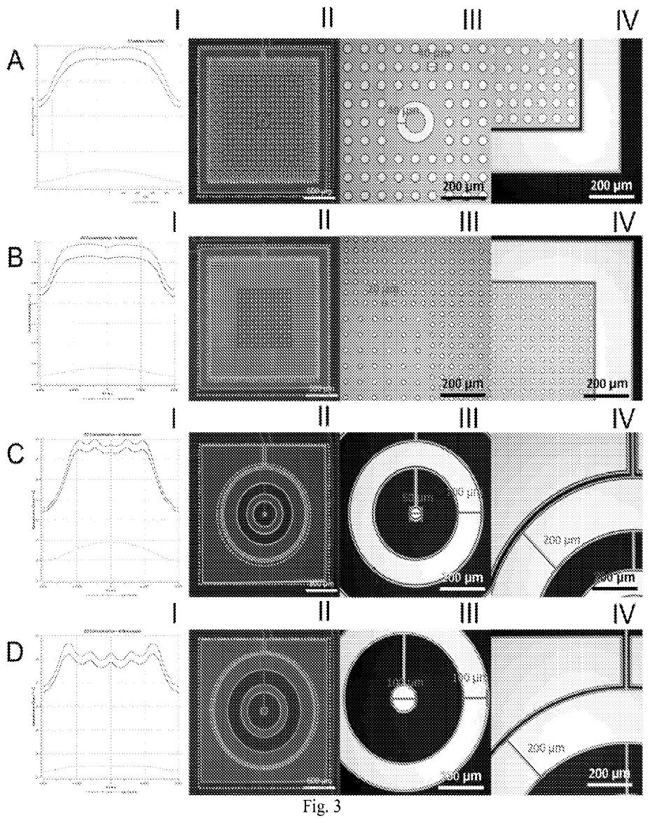

FIG. 3 shows embodiments of fine-tuned simulation-guided electrode designs with two-point source grid topologies (A) (0.0071 cm²), (B) (0.0043 cm²), and two concentric ring topologies (C) (0.0101 cm²), (D) (0.0080 cm²). The diffusion profiles of oxygen showed excellent uniformity, which is important because the dissolved oxygen is delivered to the cells generally by means of diffusion. Rows A-D of FIG. 3 show the oxygen diffusion profiles of different designs in Col. I, electrode geometries in Col. II, and magnified features with dimensions in Col. III, and IV.

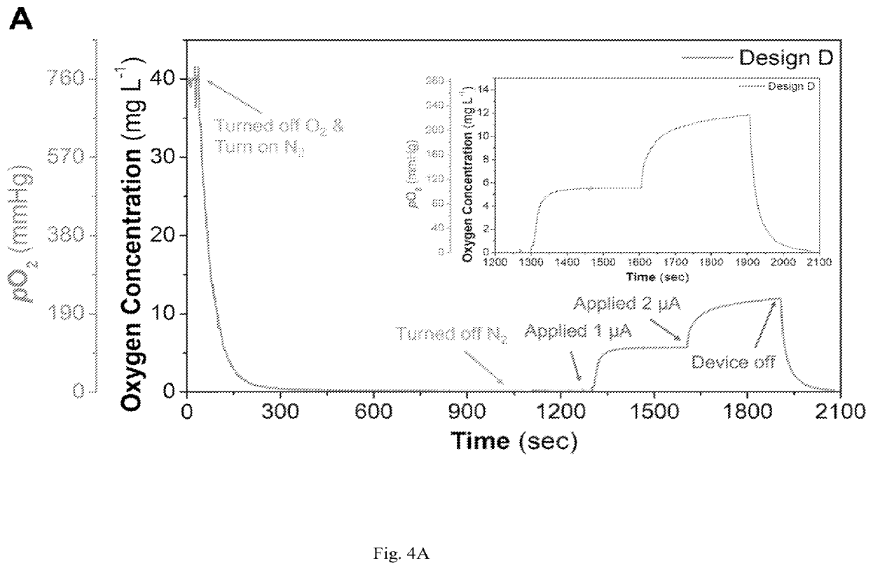

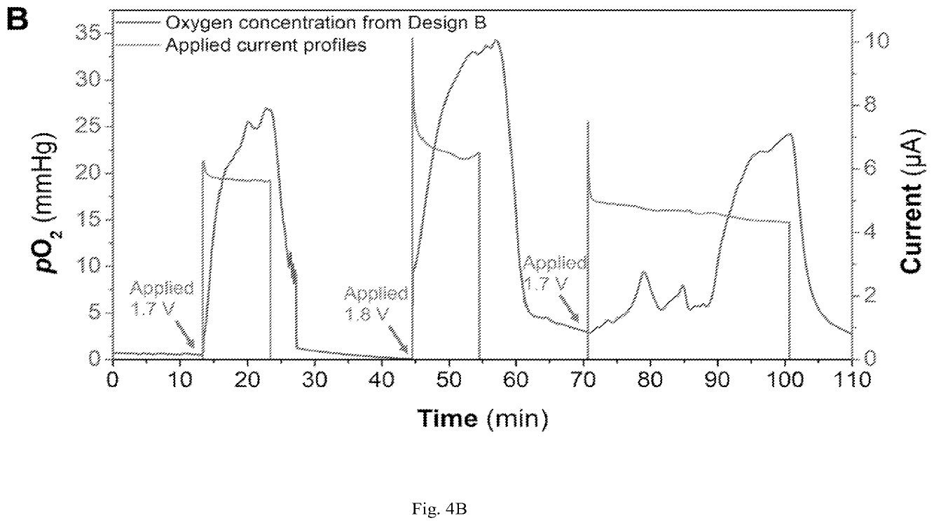

As shown in FIGS. 4A-B, actual oxygen generation behaviors of the designed electrodes B and D of FIG. 3 were validated by a Clark-type oxygen probe. FIG. 4A shows the oxygen generation profile of the design D electrode having concentric ring topologies and a size of about 0.0080 cm². FIG. 4B shows the oxygen generation profile of the design B electrode having two-point source grid topologies and a size about 0.0043 cm². In both FIGS. 4A-B, the oxygen concentration was synchronized with the applied currents, and the concentration of the oxygen was proportional to the strength of the applied current.

An undesirable outcome of the intended use of the oxygenation system is the electrochemical evolution of chlorine gas Cl₂ at the anode due to the coupling between chlorine and oxygen-binding intermediates. An important aspect of the present invention, therefore, is the design of an electrochemical oxygen generator that prevents the generation of Cl₂ gas with the help of the semipermeable membrane 110. More specifically, an ion selective membrane is used for preventing the generation of Cl₂. In one embodiment, nafion is used to suppress the activity of Cl⁻ ions near the anode surface leading to high selectivity of O₂. Nafion is a cation conducting, anion blocking fluoropolymer-copolymer that concomitantly suppresses Cl⁻ ion pathways while preserving H₂O activity near anode. In another embodiment, materials which are commonly known as having similar function to nafion are used for the semipermeable membrane 110.

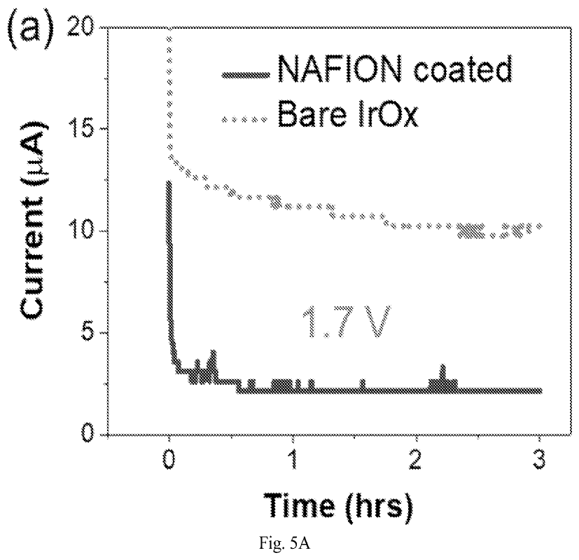

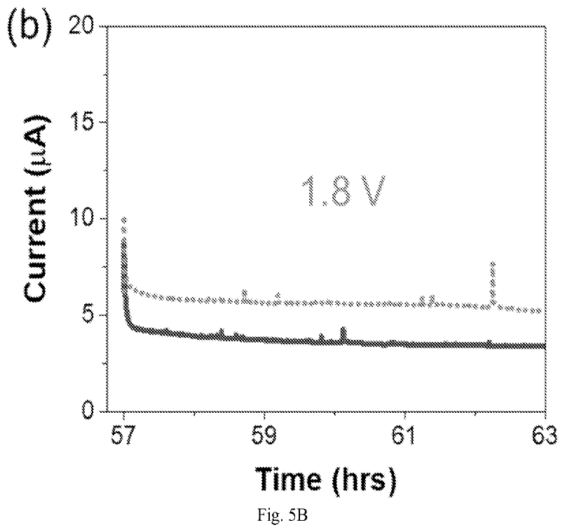

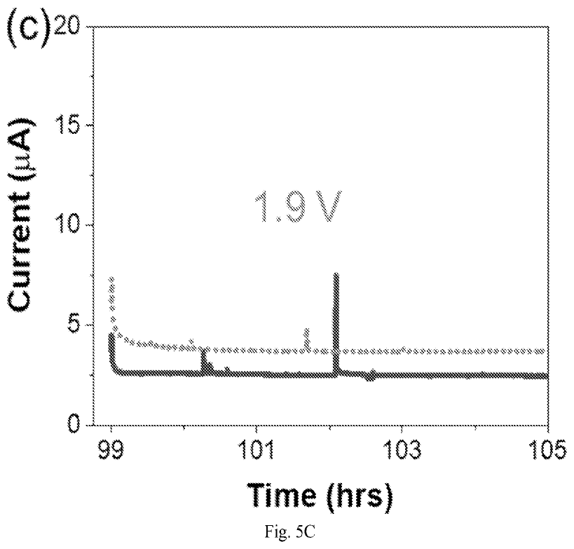

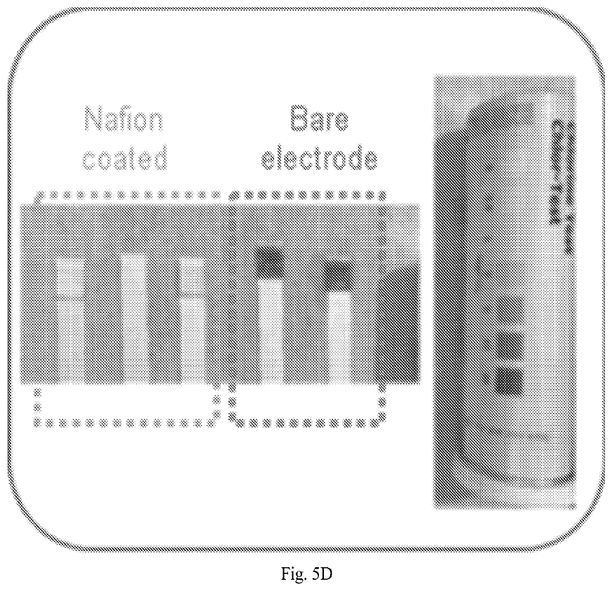

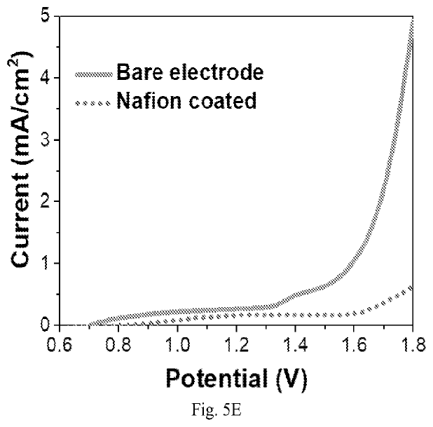

FIGS. 5A-E illustrate an example of ion-selective polymeric membrane applied electrode. LSV comparison of topology A before and after coating nafion layer demonstrates the effect of the coating on electrochemical OER characteristics. Though nafion-coated electrodes show higher impedance compared to bare electrodes, as shown in FIG. 5E, they nevertheless exhibit excellent oxygen production. FIG. 5D shows a chlorine test, which reflects that, comparing to bare electrode, the suppression of chlorine evolution is achieved in the nafion coated samples above 2V. It should be further noted that local pH changes and hydrogen peroxide production are not measurable, and does not affect the application as envisioned.

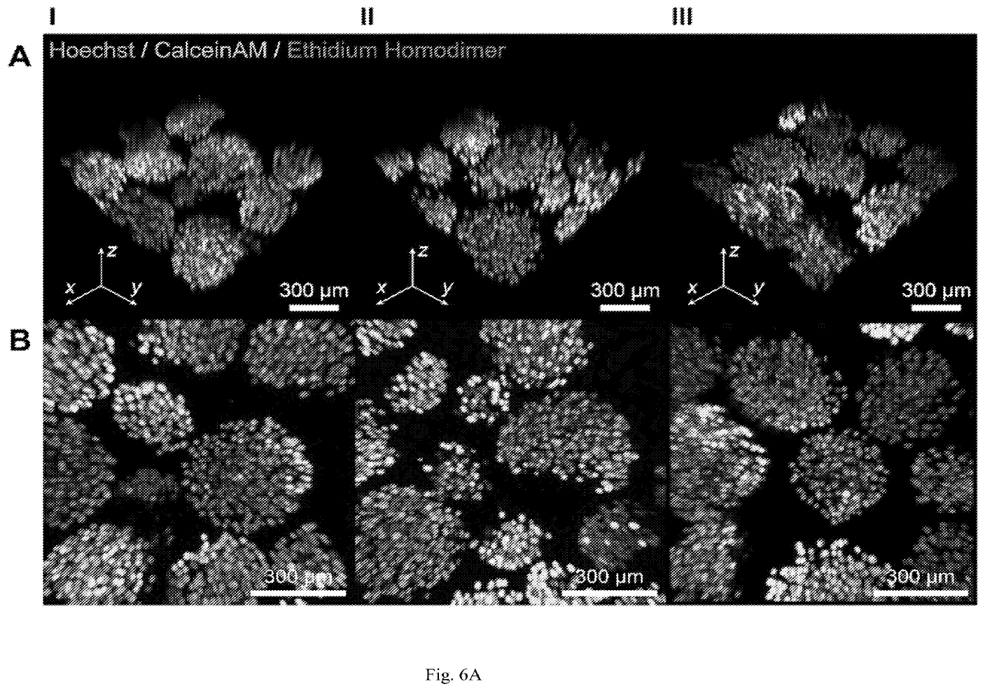

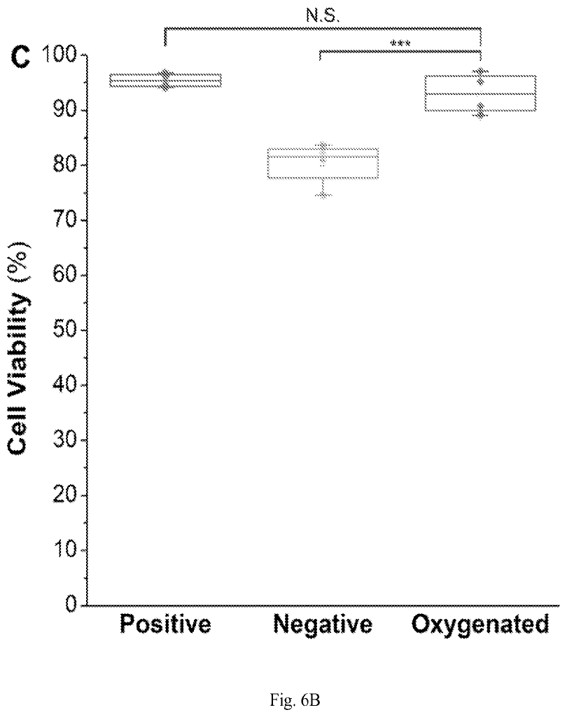

FIG. 6A shows 3D reconstruction of z-stacked images in the upper panel, and their maximum intensity projection images in the lower panel. Col. I shows positive control (normoxia), Col. II shows negative control (hypoxia), Col. III shows hypoxia+electrocatalytically oxygenated samples. As it can be seen that in the Col. I and III, the cells stained by Hoechst and Calcein AM, which specifically stain live cells but not dead cells, significantly outnumber the cells stained by Hoechst and Calcein AM in the Col. II. In contrast, the cells stained by Ethidium Homodimer, which specifically stains dead cells but not live cells, in the Col. II outnumber the cells stained by Ethidium Homodimer in the Col. I and III.

As shown in FIG. 6B, the in vitro oxygenation under hypoxia conditions shows the feasibility of cell maintenance by the electrochemical oxygen supply. At 1% O₂ (7.6 Torr) for 3 days, the oxygenated cells demonstrated significantly higher viability, compared to those that were in negative control. In contrast, there was no significant difference of the cell viability between the positive control and the oxygenated condition, bolstering the effectiveness of the oxygenation via electrochemical OER without harmful side products in the present invention.

Performance of the present invention is further improved by adjusting the duty cycles and therefore controlling the oxygenation by electrical potentials and/or currents.

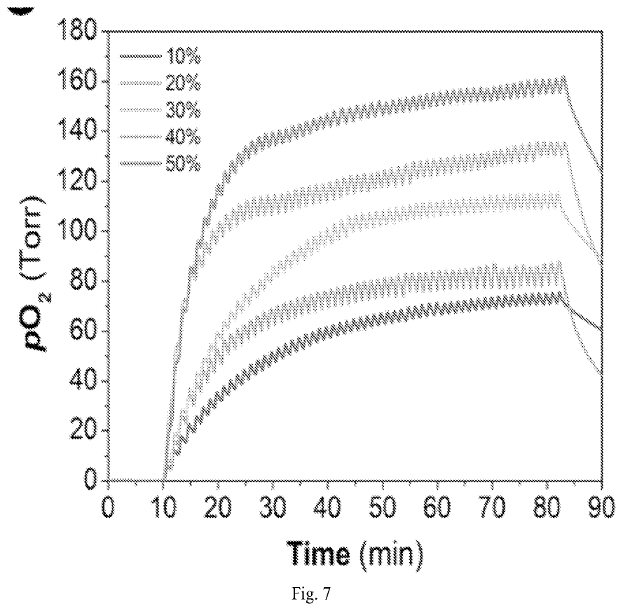

As shown in FIG. 7, partial pressure of the oxygen produced varies at different duty cycles. The present invention quantifies the oxygen concentration at various duty cycles of pulsed voltammetry so as to improve the device's lifetime and reduce the device's power consumption. FIG. 7 shows that precise control over the oxygenation is achieved by changing the duty cycles at 10, 20, 30, 40 and 50% at 1.7V. In particular, the partial oxygen pressure increases with the increasing of the percentage of the duty cycle. When the total volume is 1,500 μL and the applied potential is 1.7V, 10% duty cycle achieves a partial oxygen pressure about 70 Torr, while, at a duty cycle of 50%, the partial oxygen pressure is achieved as high as about 160 Torr. Under all duty cycles, sufficient amount of oxygen was generated to support cells in hypoxia without deterioration of the OER onset.

In one embodiment, to precisely measure the oxygen concentration at various operating conditions of the device, e.g. different duty cycles, the present invention includes an oxygen sensor. In one embodiment, the oxygen sensor is an optical sensor. The oxygen sensor works on a principle of changing the detector's optical properties relative to the oxygen concentration. In one embodiment, the oxygen sensor was placed about 500 μm above the electrodes for measurements.

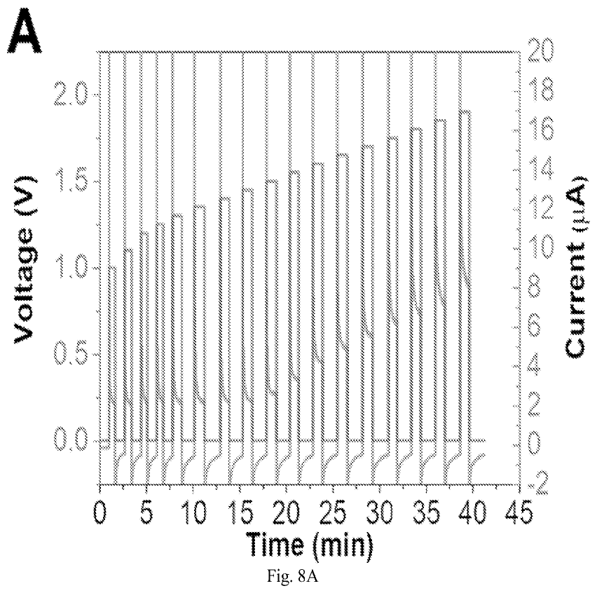

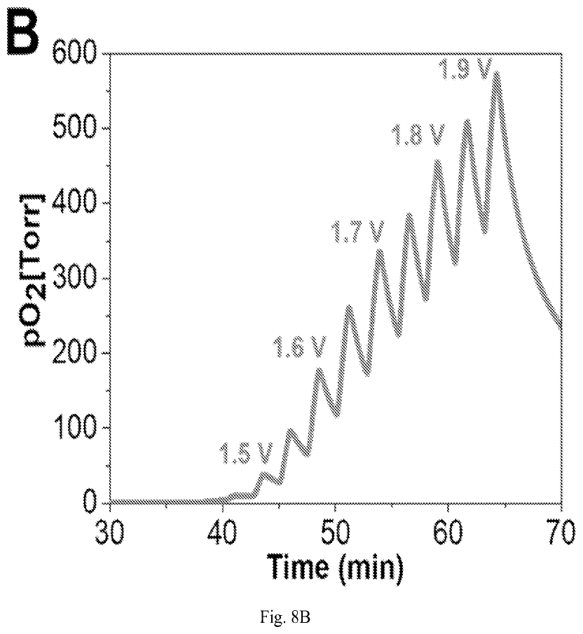

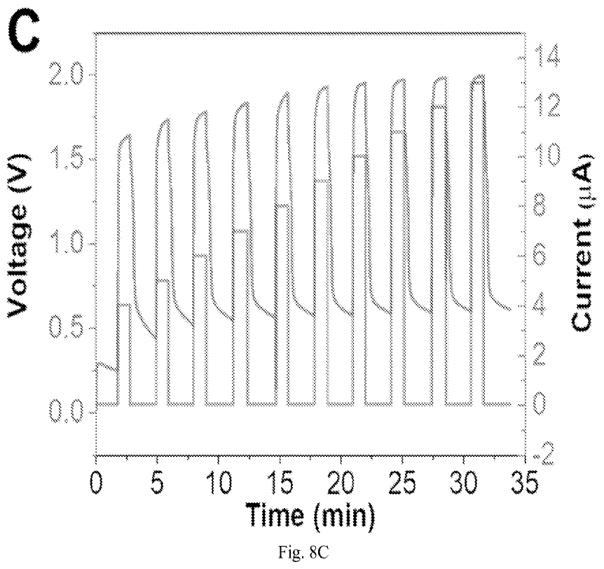

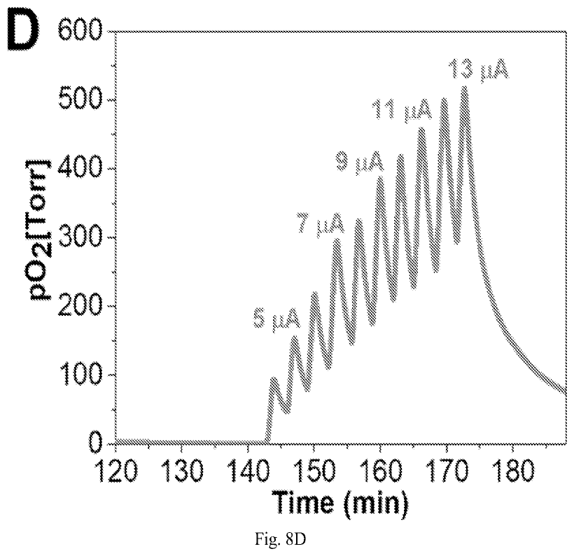

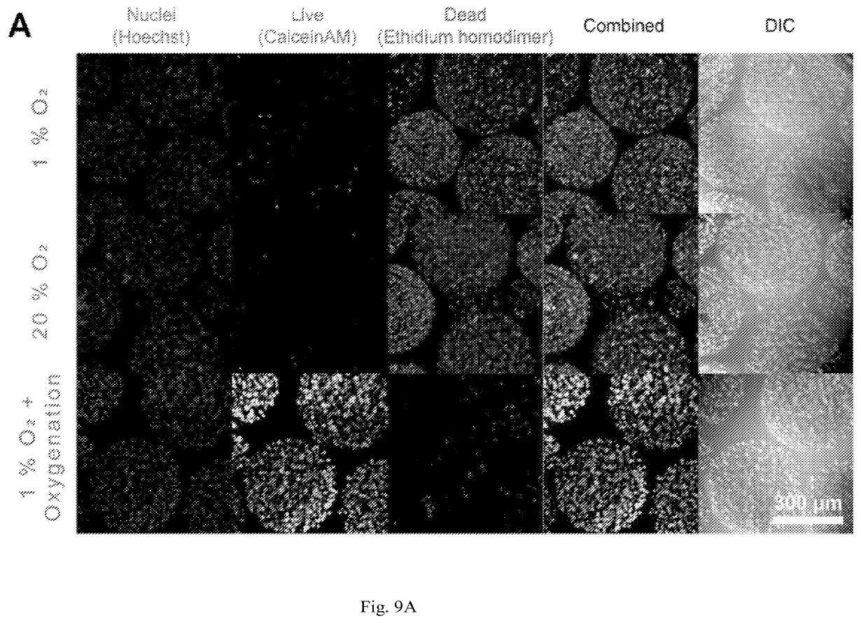

FIGS. 8A-D show the change in oxygen concentration for various two-electrode chronoamperometric pulses for SIROF electrodes. In particular, FIG. 8A shows measured current profiles for various pulsed chronoamperometry voltages, and FIG. 8B shows the oxygen concentration vs time at the various applied potentials applied in the FIG. 8A. FIG. 8C shows measured voltage profiles for various pulsed chronopotentiometry currents, and FIG. 8D shows oxygen concentration vs time at the various applied currents applied in FIG. 8C. In particular, oxygenation is detectable at potentials beyond 1.45 V albeit at very low concentration at an overpotential of merely ˜220 mV. In one embodiment, the high surface area of sputtered iridium oxide films significantly enhances the oxygenation capability of the SIROF electrodes.

As shown in FIGS. 8A-D, the oxygenation profile presents excellent controllability. As a result, the oxygen release can be precisely controlled by voltage pulsing for maintaining desired oxygen concentrations in the electrolyte.

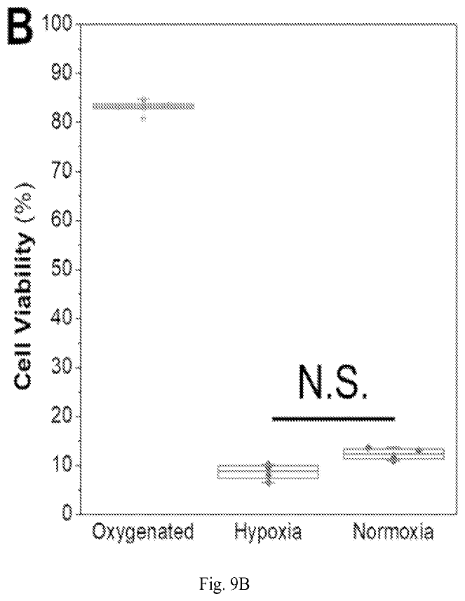

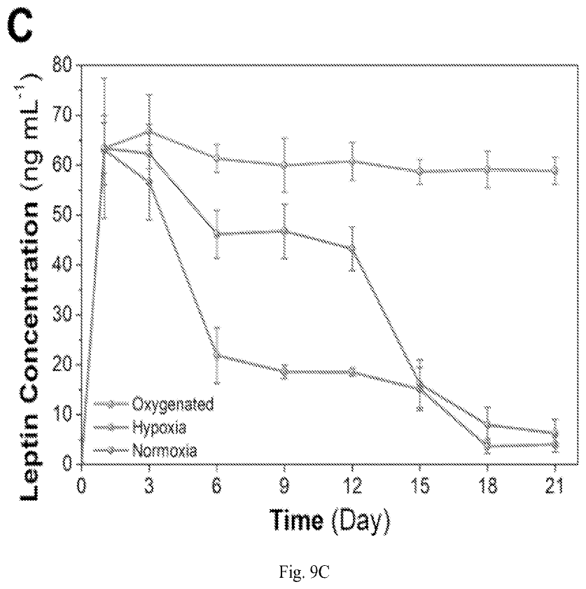

FIGS. 9A-C shows the stability of a SIROF-based oxygenator in in vitro studies. In one embodiment, the electrode of the present invention maintains cellular viability for 21 days in an oxygen-deficient environment (1% O₂) via electrocatalytic OER. According to FIGS. 9A-C, cell cultivations under normoxic (20% (2) and hypoxic (1% O₂) conditions without oxygenation showed cell viabilities of 12.31% and 8.57%, respectively. In contrasr, the oxygenated cells incubated in 1% oxygen with in vitro oxygenation for 21 days showed a significantly higher cell viability of 83.05%.

FIG. 9A shows a representative set of fluorescence MIP images of ARPE-19 cells in alginate capsules after 21-day in vitro oxygenation. Cell density was 60 k cells mm⁻³. FIG. 9B further illustrates FIG. 9A by showing the box plot of the cellular viability, while FIG. 9C shows secreted leptin concentration from the ARPE-19 cells as a function of time. It should be noted that the measured values corresponds to the amount of the leptin produced within 24 hours.

In particular, as validated by FIGS. 9A-C, in situ OER was capable of sustaining a peptide-secreting function of the ARPE-19 cells in the alginate capsules. The present invention has shown that the ARPE-19 cells only maintained their leptin-producing bioactivity with the in vitro oxygenation, while the ARPE-19 cells lost the functionality after 21 days even in normoxic cultivation. The relevant results are displayed in FIGS. 9A-C and Table 1.

TABLE 1
ARPE-19 cell viability after 21-day in vitro
oxygenation. Cell density was 60k cells mm⁻³

| Oxygen condition | |
|---|---|
| (The total number of capsules, sample size) | Cell viability (% ± σ) |
| Normoxia (81.5, n = 4) | 12.31 ± 8.96 |
| Hypoxia (83, n = 4) | 8.57 ± 5.58 |
| Hypoxia with oxygenation (102.5, n = 5) | 83.05 ± 7.54 |

In one embodiment, the oxygenation system, instead of being used in combination with an implantable or wearable in vitro device for providing oxygen to any therapeutic cells, is used in combination with an engineered tissue so as to provide oxygen to the engineered tissue.

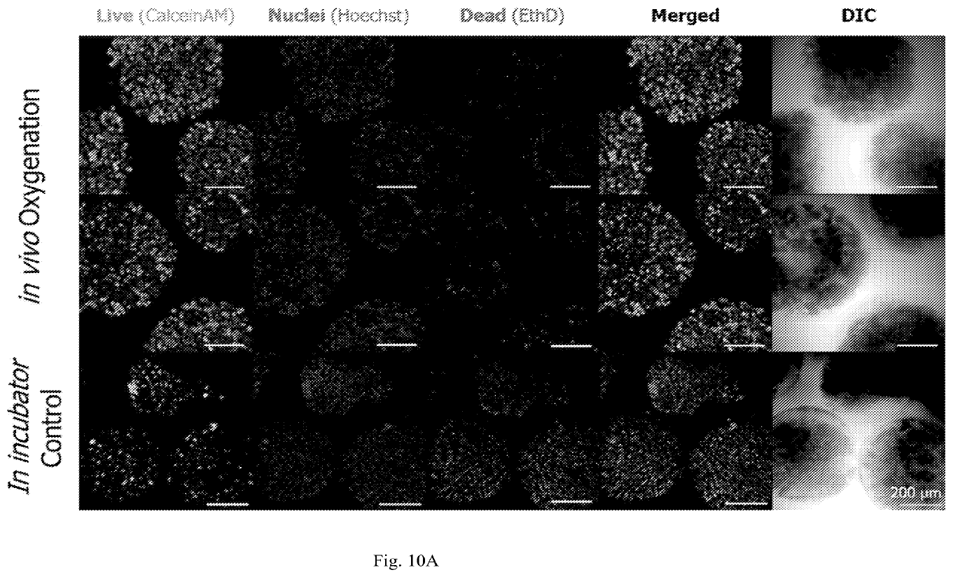

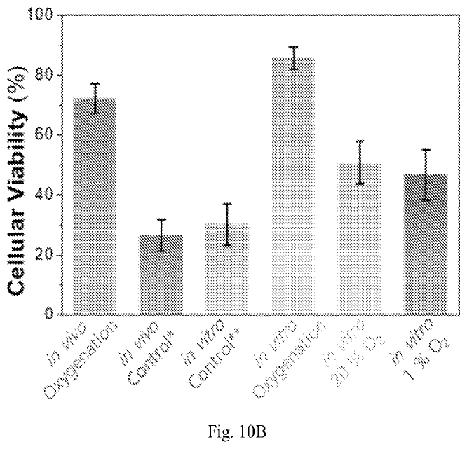

FIGS. 10A-B reflect the use and the stability of the SIROF-electrode oxygenator in in vivo studies. In one embodiment, the electrode of the present invention was implanted in the abdominal cavity of a rat model with 60 k cells mm⁻³ density alginate cell capsules (φ=300 μm), and in vivo oxygenation was carried out in 260 μL media. In particular, an oxygenator chip and cells were enclosed in medical grade PDMS, while the top of the devices was finished using a selectively permeable PCTE membrane with a pore size of 400 nm for nutrient support other than oxygen from the host vasculatures. As shown in FIG. 10A, 1st and 2nd rows display the live/dead cell staining assay results of the cell implantation with the in vivo oxygenatation, while 3rd row shows live/dead cell staining assay of a control, which conducts the cells incubation under normal oxygen level. All scale bars are 200 μm.

FIG. 10B and Table 2 show the statistics of the live/dead cell staining assay results using in vivo oxygenation and in vivo incubator control, respectively. As it can be seen, the in vivo oxygenation shows significantly improved viability of 72.24±4.96%, comparing to 26.57±5.32% of the in vivo control implantation without oxygenation (total volume: 144 μL) and 30.21±6.85% of the in-incubator control (total volume: 260 μL). The in vitro results are from 10-day in vitro oxygenation and its control, cultivated in 20% and 1% oxygen, respectively.

TABLE 2
Statistics of the live/dead assay results using
in vivo oxygenation and in vivo incubator control

| Oxygenation condition | Viability (± STD, %) |
|---|---|
| in vivo Oxygenation | 72.24 ± 4.96 |
| in vivo Control* | 26.57 ± 5.32 |
| in vitro Control** | 30.21 ± 6.85 |
| in vitro Oxygenation | 85.74 ± 3.74 |
| 20% Oxygen (in vitro) | 50.87 ± 7.05 |
| 1% Oxygen (in vitro) | 46.77 ± 8.35 |

*The volume of media: 144 μL
**Incubated in 20% oxygen within 260 μL media

The foregoing description of the exemplary embodiments of the invention has been presented only for the purposes of illustration and description and is not intended to be exhaustive or to limit the invention to the precise forms disclosed. Many modifications and variations are possible in light of the above teaching.

While there has been shown several and alternate embodiments of the present invention, it is to be understood that certain changes can be made as would be known to one skilled in the art without departing from the underlying scope of the invention as is discussed and set forth above and below including claims and drawings. Furthermore, the embodiments described above and claims set forth below are only intended to illustrate the principles of the present invention and are not intended to limit the scope of the invention to the disclosed elements.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in the description of this invention, are incorporated herein by reference in their entireties and to the same extent as if each reference was individually incorporated by reference.

What is claimed is:

1. An oxygenation system for producing oxygen for therapeutic cells housed in a cell containment system, the cell containment system having a house containing the therapeutic cells and a nutrition solution comprising water, the oxygenation system comprising:
a first electrode comprising a first material layer stack and a catalyst film or catalyst particles of a high surface area on which at least one catalyst for electrocatalytic oxygen evolution reaction (OER) is disposed, wherein the first material layer stack has a series of transition metal layers stacked on one another, or a nanocarbon layer disposed on the stacked transition metal layers, and the catalyst film or catalyst particles are disposed on a top of the first material layer stack and vertically overlap with the first material layer stack; and
a second electrode comprising a second material layer stack, wherein the second material layer stack has a series of transition metal layers stacked on one another,
wherein the first electrode is coupled with the house and the second electrode is disposed in adjacent to the first electrode;
wherein the catalyst for the electrocatalytic OER has at least one of ruthenium, iridium oxides, cobalt-based materials, nickel-based materials, organic-inorganic hybrids, and metalloprophyrin; and
wherein the catalyst is at least partially in contact with the water of the nutrition solution.

2. The oxygenation system according to claim 1, further comprising a semi-permeable selective membrane disposed at an interaction surface between the first electrode and the nutrition solution.

3. The oxygenation system according to claim 2, wherein the semi-permeable selective membrane selectively permits the passage of water and oxygen and prevents the evolution of chloride.

4. The oxygenation system according to claim 2, wherein the first electrode and the second electrode have a design of two-point source grid topology.

5. The oxygenation system according to claim 2, wherein the first electrode and the second electrode have a design of two concentric ring topology.

6. An oxygenation system for producing oxygen for therapeutic cells housed in a house of a cell containment system, the oxygenation system comprising:
a first electrode comprising a first material layer stack, and a catalyst film or catalyst particles of a high surface area on which at least one catalyst for electrocatalytic oxygen evolution reaction (OER) is disposed; and
a second electrode comprising a second material layer stack,
wherein the first electrode is coupled with the house and the second electrode is disposed in adjacent to the first electrode.

7. The oxygenation system according to claim 6, wherein the first material layer stack comprises a series of transition metal layers stacked on one another, or a nanocarbon layer disposed on the stacked transition metal layers.

8. The oxygenation system according to claim 6, wherein the second material stack comprises a series of transition metal layers stacked on one another.

9. The oxygenation system according to claim 6, wherein the catalyst film or catalyst particles are disposed on a top of the first material layer stack and vertically overlaps with the first material layer stack partially or fully.

10. The oxygenation system according to claim 6, wherein the catalyst for the electrocatalytic OER comprises at least one of ruthenium, iridium oxides, cobalt-based materials, nickel-based materials, organic-inorganic hybrids, and metalloprophyrin.

11. The oxygenation system according to claim 6, wherein the catalyst is in contact with water of nutrition solution.

12. The oxygenation system according to claim 11, further comprising a semi-permeable selective membrane disposed at an interaction surface between the first electrode and the nutrition solution.

13. The oxygenation system according to claim 12, wherein the semi-permeable selective membrane selectively permits the passage of water and oxygen and prevents the evolution of chloride.

14. The oxygenation system according to claim 6, wherein a duty cycle of at least one of the first and second electrodes is adjustable.

15. The oxygenation system according to claim 6, wherein the first electrode and the second electrode have a design of two-point source grid topology.

16. The oxygenation system according to claim 6, wherein the first electrode and the second electrode have a design of two concentric ring topology.

17. An implanted or external device for housing therapeutic cells and producing oxygen for the therapeutic cells, the implanted or external devices comprising:
a cell containment system having a house containing the therapeutic cells and a nutrition solution comprising water; and
an oxygenation system,
wherein the oxygenation system has at least one electrode coupled with the house.

18. The implanted or external device according to claim 17, wherein at least one electrode comprises a first electrode and a second electrode.

19. The implanted or external device according to claim 18, wherein the first electrode comprises a catalyst film or catalyst particles of a high surface area on which at least one catalyst for electrocatalytic oxygen evolution reaction (OER) is disposed.

20. The implanted or external device according to claim 19, wherein the first electrode comprises a first material layer stack.

21. The implanted or external device according to claim 20, wherein the first material layer stack having a series of transition metal layers stacked on one another, or a nanocarbon layer on of the stacked transition metal layers.

22. The implanted or external device according to claim 21, wherein the second electrode comprises a second material layer stack having a series of transition metal layers stacked on one another.

23. The implanted or external device according to claim 20, wherein the catalyst film is disposed on a top of the first material layer stack and vertically overlaps with the first material layer stack partially or fully.

24. The implanted or external device according to claim 19, wherein the catalyst for the electrocatalytic OER comprises at least one of ruthenium, iridium oxides, cobalt-based materials, nickel-based materials, organic-inorganic hybrids, and metalloprophyrin.

25. The implanted or external device according to claim 17, wherein the catalyst is in contact with water of the nutrition solution.

26. The implanted or external device according to claim 17, wherein the oxygenation system further comprises a first semi-permeable selective membrane disposed at an interaction surface between the first electrode and the nutrition solution.

27. The implanted or external device according to claim 26, wherein the first semi-permeable selective membrane selectively permits the passage of water and oxygen, and prevents the passage of cells, macromolecules, and salts,
wherein the salts contain chloride ions.

28. The implanted or external device according to claim 27, further comprising a second semi-permeable selective membrane disposed at an interface between the implanted or external device and body environment of a user.

29. The implanted or external device according to claim 28, wherein the second semi-permeable selective membrane prevents passage of the immune cells of the user into the house.

30. The implanted or external device according to claim 17, wherein the oxygenation system comprises an insulating layer protecting the at least one electrode.

31. The implanted or external device according to claim 17, wherein the oxygenation system comprises a flexible substrate on which the at least one electrode is disposed.

32. The implanted or external device according to claim 17, wherein the therapeutic cells are encapsulated in a 3D morphology or hydrogel.

33. The implanted or external device according to claim 17, wherein a density of the therapeutic cells exceeds 50 k cells/mm³.

34. The implanted or external device according to claim 17, wherein
a concentration of the produced oxygen in the nutrition solution is adjustable by controlling a duty cycle of the at least one electrode.

35. The implanted or external device according to claim 17, wherein the cell containment system comprises a light source configured to produce light for the therapeutic cells; wherein the light source is configured to produce a first light and a second light, and the second light having a wavelength different from the first light.
